(19) United States
(12) Patent Application Publication
(10) Pub. No.: US 2006/0148065 A1
(43) Pub. Date: Jul. 6, 2006

(54) Collection of micro scale devices

(76) Inventor: Mats Inganas, Uppsala (SE)

Correspondence Address: FULBRIGHT & JAWORSKI, LLP, 1301 MCKINNEY, SUITE 5100, HOUSTON, TX, 77010-3095, US

(21) Appl. No.: 10/550,182

(22) PCT Filed: Mar. 23, 2004
(86) PCT No.: PCT/SE04/00441

§ 371 Date: Sep. 21, 2005

Related U.S. Application Data
(60) Provisional application No. 60/466,250, filed on Apr. 29, 2003.

(30) Foreign Application Priority Data
Mar. 23, 2003 (SE) 0300822-4

(51) Int. Cl. C12M 1/34

(52) U.S. Cl. 435/287.2

(57) ABSTRACT

A collection of one or more microfluidic devices which together carry a plurality of microchannel structures each of which comprises a reaction microcavity (104a-h) in which there is a solid phase with an immobilized affinity ligand L, characterized in that (i) the plurality is divided into sets of microchannel structures, and (ii) the affinity ligand L is directed to the same counterpart (binder, B) independent of set, and (iii) the sets differ in a) the capacity per reaction microcavity and/or the capacity/unit volume of the solid phase in a reaction microcavity for binder B, and/or b) the base matrix of the solid phase between the sets but are equal within each set.

### TECHNICAL FIELD

The present invention relates to a collection of one or more microfluidic devices, each of which carries one or more microchannel structures in which there is a reaction microcavity containing a solid phase with an immobilized affinity ligand.

Each microchannel structure is primarily intended for performing an affinity protocol utilizing an affinity reaction in which an affinity complex AC_{S}—S between a solute S and its affinity counterpart AC_{S} is formed or dissociated. The affinity counterpart AC_{S} is typically immobilized or immobilizable to the solid phase. The affinity complex may comprise other affinity reactants in addition to S and AC_{S}.

The affinity protocol may be an affinity assay such as an enzyme assay, an immune assay, a cell based assay, a hybridization assay and other affinity assays in which an uncharacterized analyte or some other reaction variable is to be characterized. The affinity reaction may also be part of a protocol for which the main objectives are separation, purification and/or enrichment.

The term “solute” includes true solutes, microorganisms such as viruses, suspended cells, suspended cell parts and various other reactants that are in dissolved or colloidal form and sufficiently small to be transported by liquid flow through the bed. The solute S may be the analyte of an original sample or an analyte-derived entity formed before the affinity reaction between the solute S and its affinity counterpart AC_{S}.

The term “microfluidic device” means that a liquid flow is used for transporting various kinds of reactants, samples, buffers etc in the microchannel structures of the device.

The term “micro” in “microchannel structure”, contemplates that the structure comprises one or more cavities and/or conduits that have at least one cross-sectional dimension that is ≦10³ μm, preferably ≦5×10² μm, such as ≦10² μm. Similarly applies also to “microconduit”, “microvolume”, microcavity etc. The volume of a liquid aliquot to be processed within a a microchannel structure is typically in the nanolitre (nl) range (which includes the picolitre (pl) range). The nl-range has an upper end of 5,000 nl but relates in most cases to volumes ≦1,000 nl, such as ≦500 nl or ≦100 nl.

Patent publications cited herein are incorporated by reference in their entirety.

### BACKGROUND PUBLICATIONS

WO 02075312 (Gyros AB) focuses on affinity assays for the characterization of reaction variables by binding an affinity reactant to a solid phase that exposes the counterpart to the affinity reactant or by releasing the affinity reactant from a solid phase that comprises an immobilized affinity complex between the affinity reactant and its affinity counterpart. Various immobilization techniques are suggested, e.g. covalent binding, physical adsorption, bioaffinity binding including streptavidin-biotin, etc.

U.S. Pat. No. 5,726,026 (Univ. Pennsylvania) and U.S. Pat. No. 5,928,880 (Univ. Pennsylvania) describe a microfluidic device that may comprise a solid phase in the form of particles to which streptavidin have been pre-bound. The particles can be sensitized with biotinylated antibody.

### BACKGROUND PROBLEMS

It would be of big economic benefit if the same or similar standard procedures could be used for the manufacture of microfluidic devices intended for a range of various assay and/or separation protocols, a number of concentration ranges, a range of various analytes etc. This includes that it is important to provide microfluidic devices that enable the measurement of analytes of low as well as of high activity and/or concentration in small volume samples, e.g. concentrations in the nanomolar range in μl-samples. In this context the nanomolar range comprises ≦1,000×10⁻⁹ M and includes the picomolar range ≦1,000×10⁻¹²M. μl-samples comprises ≦1,000 μl and includes nl-samples ≦5,000 nl.

It is often difficult and critical to find affinity reagents/reactants, such as antibodies, of a quality that will give a sufficiently high analytical sensitivity and specificity, diagnostic sensitivity and specificity etc required by diagnostic and medical needs. The affinity and the specificity of one reactant, for instance, may be difficult to match with other reactants of a protocol in order to comply with preset specifications. It would thus be beneficial to provide assay systems and methodologies that inherently increase features such as sensitivity to thereby make more affinity reagents available without compromising earlier decided specifications.

### OBJECTS OF THE INVENTION

A first main object is to provide improved microfluidic devices that solve the problems discussed above. This includes providing methods for manufacturing and use of the devices.

A second main object is to provide a product offer that is intended for performing affinity reactions of a range of different affinity reactants, such as analytes, which may be present in the same or different concentration intervals by a range of different assay protocols. The offer can be envisaged as a number of microchannel structures which are portioned into sets where each set is dedicated to a particular combination of analytes, and/or a particular range of concentrations of the same or different analytes, and/or particular combinations of different affinity protocols.

A third main object is to provide a system for affinity assays that will expand the range of affinity reactants/reagents that will fulfill performance requirements on analytical and diagnostic sensitivity and specificity of an affinity assay.

In this context the term “different affinity protocols” refers to a number of affinity protocols each of which differ with respect to at least one member selected amongst (a) the reactants used, (b) the sequence of steps, and (c) incubation times, for instance contact time with a solid phase, from one or more of the other protocols.

### DRAWINGS

FIG. 1 gives a subgroup of microchannel structures of the microfluidic device utilized in the experimental part.

FIG. 2 shows the dynamic capacity for different kind of solid phase material in particulate form: Superose™ 20 mg/ml wash with aqueous isopropananol (IPA) (graph 1), Superose™ 2 mg/ml (graph 2), Superdex™ P wash with IPA (graph 3), Polystyrene-PheDex (graph 4), Sepharose™ HP wash with IPA (graph 5). Fluorescence intensity is given in the radial direction through the bed (length of the bed) with the peak corresponding to the solute typically at the entrance. The flow direction is from the right to the left. Affinity ligand L is streptavidin which is used to sensitize the solid phase material with an assay component (in this case anti-myoglobin antibody). The effect is measured in the fluorescence myoglobin immunoassay given below with a concentration of myoglobin of 500 nM.

FIG. 3 shows the result with a solid phase materials having different binding capacities: 5.5 mg streptavidin/ml porous bed (graph 1), 0.58 mg streptavidin/ml porous bed (graph 2), and 0.08 mg streptavidin/ml porous bed (graph 3). Measurement as for FIG. 2. The trailing peak for graph 3 is caused by unwanted binding labeled antibody to the solid phase.

### THE INVENTION

We have discovered that an affinity captured solute may appear as a more concentrated band in the solid phase if the capturing reactant AC_{S} is affinity immobilized compared to covalently immobilized. This suggests a more efficient capture and an increased analytical sensitivity if the capturing is part of an affinity assay.

Indications of increased analytical sensitivity for affinity immobilization have also been deduced from dose-response graphs.

We have realized that our discovery may have an impact on the selection of immobilization technique if one would like to provide a product offer as defined for the second main object of the invention.

Based on our discovery we have recognized that assaying of analytes of high as well as of low concentrations can be accomplished with high accuracy by using the proper combination of:
a) Flow conditions during the capturing step, i.e. during the formation of the immobilized complex AC_{S}—S.
b) A generic immobilizing binding principle based on a common affinity ligand (L, e.g. streptavidin) immobilized to the solid phase and/or one or more different conjugates B-AC_{S1}, B-AC_{S2} etc where B is a common binder (immobilizing binder B, e.g. biotin) directed towards the common ligand L and B-AC_{S1}, B-AC_{S2} etc are affinity counterparts directed towards different solutes (S1, S2 etc).
c) Selection of the binding capacity of the solid phase for the conjugate (or for the immobilizing binder in unconjugated form) and saturation of the solid phase with a conjugate that is capable of capturing a particular solute S.
d) Selection of the matrix of the solid phase material.
e) Balancing the flow conditions (flow rate and/or residence time) in relation to the affinity pair (AC_{S} and S) to achieve non-diffusion limiting conditions for optimal enrichment of the solute on the solid phase.

The capacity per reaction microcavity and/or the capacity per volume unit of the solid phase in a reaction microcavity for binding the conjugates (e.g. biotin conjugates) will determine the optimal
a) kind(s) of solute(s) and analyte(s),
b) concentration range of solute/analyte, and/or
c) assay protocols etc, for a particular solid phase/microchannel structure.

First Aspect: Collection of Microdfluidic Devices

The main aspect of the invention is a collection of one or more microfluidic devices which together carry a plurality of microchannel structures (101a-h) each of which comprises a reaction microcavity (104a-h) in which there is a solid phase with an immobilized affinity ligand L. The characteristic feature of the set is that
i) the plurality of microchannel structures comprises two or more different sets of microchannel structures, and
ii) the affinity ligand L is directed to the same counterpart (binder, B) independent of set, and
iii) the sets differ from each other in that
   a) the capacity per reaction microcavity, and/or the capacity per unit volume of the solid phase in a reaction microcavity (104a-h) for binder B, and/or
   b) the base matrix of the solid phase material differ between the sets but are equal within each set.

Each of the sets is intended for a particular affinity protocol/particular affinity protocls that may or may not differ for different sets. These protocols may differ with respect to the reactants involved and/or the order of addition of the reactants and/or the concentration range in which the reactants are used. Each of the different protocols utilizes an affinity reaction between
a) a solute S, and
b) a conjugate that comprises (i) binder B, and (ii) an affinity counterpart AC_{S} to the solute S (conjugate=B-AC_{S}).

The solute S and thus also the affinity counterpart AC_{S} may differ between the protocols.

Collection of Microfluidic Devices Versus Sets of Microchannel Structures.

A microfluidic device of the innovative collection comprises
a) microchannel structures of at least two different sets, and/or
b) microchannel structures of only one set.

Alternative (b) does only apply if there are two or more of the microfluidic devices in the collection and that these two devices comprise different sets as defined above.

The number of microchannel structures representing a set on a single microfluidic device is typically two, three or more.

The collection may contain microfluidic devices that are multiplicates, i.e. several devices that have the same combination of sets but differ in other respects, e.g. (1) total number of microchannel structures, and/or (2) the number of microchannel structures of a particular set, (3) grouping and/or positioning of groups/microchannel structures on a device (see below), (4) combination of functional units, (5) number of microchannel structures that do not have an affinity ligand L, (6) etc.

On a microfluidic device that comprises microchannel structures of several sets, those of the same set are preferably placed in one or more groups with each group being located to a particular area/subarea of the device. The microchannel structures within the same group and/or set are preferably fluidly equivalent. On a centrifugally based microfluidic device, for instance, the microchannel structures of a set or a group may be located in a defined sector or in the same annular zone. The corresponding parts of the microchannel structures of a group are then preferably at the same radial distance from the spin axis of the device.

The term “fluidly equivalent microchannel structures” means that the structures have the same combination of fluid functions and/or can be processed in parallel by a commonly applied force for driving a liquid flow through them.

In preferred variants, each of the reaction microcavitity(ies) (104a-h) which contains the solid phase with an affinity ligand L communicates in the upstream direction with a volume-metering unit (106a-h,108a-h). Each volume-metering unit is typically part of an inlet arrangement (102,103a-h) and comprises an inlet port (105a-b,107a-h) for receiving liquid and/or particles that are dispensed to the device. The volume-metering units (106a-h,108a-h) for two or more microchannel structures (101a-h) of the same set and/or the same group on the same device, may define a distribution manifold that is common for these microchannel structures (subset/subgroup). This kind of distribution manifold may or may not be part of a common inlet arrangement (102) with one or more inlet ports (105a-b) that typically are common for the same microchannel structures (101a-h) as the common inlet arrangement and/or as the distribution manifold. A volume-metering unit/inlet arrangement (108a-h/103a-h) may alternatively be linked to only one microchannel structure/reaction microcavity (101a-h/104a-h). A microchannel structure (101a-h) in the innovative collection may thus have either one or both of the above-mentioned types of inlet arrangements (102/103a-h). Each group of microchannel structures (101a-h) defined by a common inlet arrangement and/or distribution manifold (102a-b/103a-h) is preferably located to a particular subarea of the device.

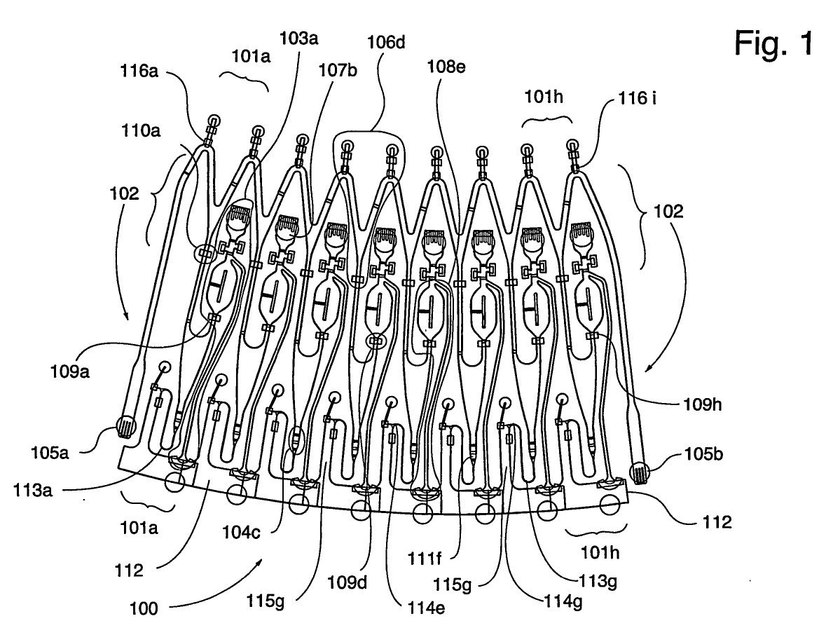

Each volume-metering unit (106a-h,108a-h) typically has a valve at its outlet end (109a-h,110a-h). This valve is typically passive, for instance utilizing a change in surface characteristics at the outlet end, such as a boundary between a hydrophilic and hydrophobic surface (hydrophobic surface break). See FIG. 1 and the experimental part. Useful inlet arrangements with volume-metering units, distribution manifolds, inlet ports and valves have been presented in WO 02074438 (Gyros AB), WO 02075312 (Gyros AB), WO 02075775 (Gyros AB) and WO 02075776 (Gyros AB).

Positioning of the microchannel structures (101a-h) of a device into groups may also be according to criteria other than sets and/or common inlet functions. Grouping may for instance be according to other common functions, such as common waste functions, common venting functions, type of mixing function, type of heating function etc. See below.

The number of sets of microchannel structures of the innovative collection is typically 2, 3, 4,5, 6, 7 . . . 10 or more.

In addition to the difference in binding capacity and/or base matrix, the sets may also have other differences, for instance with respect to other functional units and how they are connected.

Immobilizing Affinity Pair (L and B)

The affinity ligand L and its counterpart (=binder B) are called an immobilizing affinity pair. Typically the immobilizing binding pair should be selected such that, except for the desired affinity binding to each other, they should be essentially devoid of other binding abilities during the conditions used, for instance towards other reactants.

Preferred immobilizing affinity pairs (L and B) typically have affinity constants (K_{L-B=[L][B]/[L-B]) that are ≦}10 times or ≦10² times or ≦10³ times larger than the corresponding affinity constant for streptavidin and biotin. This typically will mean affinity constants that roughly are ≦10⁻¹³ mole/I, ≦10⁻¹² mole/I, ≦10⁻¹¹ mole/I and —10⁻¹⁰ mole/I, respectively.

It is believed that it is advantageous that the immobilized ligand L has two or more binding sites for the immobilizing binder B, and/or the immobilizing binder B has one, two or more binding sites for the ligand L (or vice versa).

Particular examples of immobilizing affinity pairs are a) streptavidin/avidin/neutravidin and a biotinylated reactant (or vice versa), b) antibody and a haptenylated reactant (or vice versa), c) an IMAC group and an oligopeptide containing a sequence of histidyl and/or histidyl residues (i.e. an IMAC motif) linked to a reactant, etc.

The preference is to select L and B amongst biotin-binding compounds and streptavidin-binding compounds, respectively, or vice versa. Streptavidin and neutravidin are believed to work best as the immobilized affinity ligand L.

The affinity ligand L should be attached more firmly to the solid phase than the binder B is affinity bound to the affinity ligand L. This is valid for the conditions applied during binding of the solute S to the solid phase via the immobilizing binding pair, i.e. via the conjugate B-AC_{S}. This typically means covalent immobilization of the affinity ligand L although also adsorptive bonds involving electrostatic attraction, van-der Waals bonding etc may be used. Utilizing polymeric carriers carrying both the affinity ligand and a plurality of groups that are capable of interacting with a counter structure on the surface of the solid phase may accomplish strong attachment of the affinity ligand L via adsorptive forces, for instance.

The binding capacity of the solid phase for binder B can be measured as the amount of affinity ligand L in mole per unit volume, disregard blocking and destruction of binding sites caused by the immobilization. With this measure suitable binding capacities will typically be found within the interval of 0.001-3000 pmole, such as 0.01-300 pmole, per nl solid phase in bed form saturated with liquid. For instance, if 0.1 pmole streptavidin per nl has been immobilized this corresponds 0.4 pmole/nl biotin-binding sites. The conversion factor four is because streptavidin has four binding sites for biotin per streptavidin molecule.

Binding capacity can also be measured as actual binding capacity for binder B, i.e. mole active binding sites per unit volume of the solid phase containing the immobilized affinity ligand in bed form saturated with liquid. This kind of binding capacity will depend on the immobilization technique, the pore sizes of the solid phase, the size of the entity to be immobilized, the material and design of the solid phase etc. Ideally the same ranges apply for the actual binding capacity as for the total amount of binding sites (as defined above).

Measurements of actual binding capacities can be carried out according to principles well known in the field. This typically means that affinity ligands of the solid phase is saturated with an excess binder molecules whereafter the amount bound is measured, for instance directly on the solid phase or after elution. To facilitate measurement labeled forms of binders may be used, for instance by the use of a mixture of labeled and unlabelled binder.

The actual binding capacity primarily refer to binding/capturing of the binder B in its basic form, e.g. unconjugated and/or underivatized.

In the case the solid phase is an inner wall of the reaction microcavity, the volume of the solid phase is taken as the volume of the reaction microcavity.

The optimal range of binding capacities (for B) for a particular kind of experiments depends on a number of factors, e.g. kind of solute and/or the original analyte and/or the concentration range in which the solute/analyte is measured, immobilizing affinity pair, kind of solid phase, e.g. porosity and its base material, size of conjugate (B-AC_{S}), ratio between number of binding structures for L and number of binding structures for S (of the conjugate), etc. Testing by trial and error is at the moment the safest way to optimize the binding capacity in relation to a particular experiment or protocol.

Binding capacities (for B) can also be measured per reaction microcavity. The intervals within which suitable binding capacities of this kind can be found are deducible from the capacity ranges given above combined with the intervals for the bed volumes given below.

The differences between the sets in binding capacity (for B) per volume unit are typically a factor ≧1.2 for at least 1, 2, 3, 4, 5, 6, 7, 8, 9, 10 etc sets, for instance for 5-95%, such as ≧10% or ≧25% or ≧40% or ≧55% or ≧70%≧85% and/or ≦85% or ≦70% ≦55% or ≦40% or ≦25% or ≦19%, of the sets of the collection compared to the binding capacity per volume unit for the set having the lowest binding capacity. The factor may also be ≧2 or ≧4, such as ≧10 or ≧50 or ≧90 or ≧500 for the same number and the percentages of the sets as when the factor is ≧1.2. Due account has to be taken so that the sum of percentages for the sets below and for the sets above a factor always add to 100%.

The same intervals for the various factors and the percentages apply also for the binding capacity per reaction microcavity/bed.

The Affinity Counterpart AC_{S} and the Solute S

The affinity counterpart AC_{S} is capable of binding to the solute S by affinity. This kind of binding is typically based on at least one of: (a) electrostatic interactions, (b) hydrophobic interactions, (c) electron-donor acceptor interactions, and/or (d) bioafinnity binding.

Bioaffinity binding is a subgroup of affinity binding that typically comprises a combination of interactions.

The affinity counterpart AC_{S} may thus:
(a) be electrically charged or chargeable, i.e. contains positively charged nitrogen (e.g. primary, secondary, tertiary or quaternary ammonium groups, and amidinium groups) and/or negatively charged groups (e.g. carboxylate groups, phosphate groups, phosphonate groups, sulphate groups and sulphonate groups); and/or
(b) comprise one or more hydrocarbyl groups and other hydrophobic groups; and/or
(c) comprise one or more heteroatoms (O,S,N), possibly linked to hydrogen and/or an sp-, sp²_ and/or sp³-hybridised carbon, and/or
(d) comprise a combination of features (a)-(c).

The affinity counterpart AC_{S} may be selected amongst reactants that are members of a bioaffinity pair. Typical bioaffinity pairs are a) antigen/hapten and an antibody, b) complementary nucleic acids, c) immunoglobulin-binding protein and immunoglobulin (for instance IgG or an Fc-part thereof and protein A or G), d) lectin and the corresponding carbohydrate, e) biotin and (strept)avidin, e) members of an enzymatic system (enzyme-substrate, enzyme-cofactor, enzyme-inhibitor etc), f) a sequence of histidyl, cysteinyl, phosphorylated aminoacyl etc residues and an IMAC group (immobilized metal chelate), etc. Antibody includes antigen binding fragments and mimetics of antibodies and their fragments and recombinant constructs. The term “bioaffinity pair” includes also affinity pairs in which one or both of the members are synthetic, for instance mimicking one or both of the members of a native bioaffinity pair.

The term “affinity reactant” also includes a reactant that is capable of reversible covalent binding with its counterpart, for instance by disulfide formation. This kind of reactants typically exhibits a HS— or a —S—SOₙ— group (n=0,1 or 2, free valences bind to carbon). See U.S. Pat. No. 5,887,997 (Batista), U.S. Pat. No. 4,175,073 (Axén et al), and U.S. Pat. No. 4,563,304 (Axén et al).

The affinity counterpart AC_{S} may also be a catalytic system or a member of a catalytic system, such as a catalyst, a cocatalyst, a cofactor, a substrate or cosubstrate, an inhibitor, a promotor etc. For enzymatic systems the corresponding members are enzyme, cocatalyst, cofactor, coenzyme, substrate, cosubstrate etc. The term “catalytic system” also includes linked catalytic systems, for instance a series of systems in which the product of the first system is the substrate of the second catalytic system etc and whole biological cells or a part of such cells.

The affinity counterpart AC_{S} should be selected to have the appropriate selectivity and specificity for binding the solute to the solid phase material in relation to an intended application. General methods and criteria for the proper selection of affinity reactants and reaction conditions are well known in the field.

The affinity constant (K_{S-AC}=[S][AC_{S}]/[S-AC_{S}]) for the formation of the complex comprising the affinity counterpart AC_{S} and the solute S is an important criterion for optimizing an application. The demand on this affinity constant varies depending on application. For affinity assays the affinity constant is typically ≦10⁻⁸ mole/I or ≦10⁻⁹ mole/I. This kind of assays typically includes that the solute is reacted with immobilized AC_{S} under flow conditions and related to the amount of an analyte in an animal or biological sample (animal or biological sample include samples from mammals, such as human and other animal patients, and from experimental animals). This does not exclude that affinity counterparts having weaker affinities may be used for this kind of samples, other samples and affinity assays, and other applications. Thus depending on application the affinity constant may be relatively large, such as up to 10⁻³ mole/I or up to 10⁻⁴ mole/I or up to 10⁻⁵ or up to 10⁻⁷ mole/I.

The affinity constants for the L/B- and AC_{S}/S-pairs refer to values obtained by a biosensor (surface plasmon resonance) from Biacore (Uppsala, Sweden), i.e. with the affinity reactant (AC_{S} and L) immobilized to a dextran-coated gold surface.

At least one of the members of an affinity pair, in particular a bioaffinity pair, typically exhibits a structure selected amongst: a) amino acid structure including peptide structure such as poly and oligopeptide structure, b) carbohydrate structure, c) nucleotide structure including nucleic acid structure, d) lipid structure such as steroid structure, triglyceride structure etc. This kind of structures may be present in L, B, AC_{S} and S as well as in other affinity reactants used.

The Conjugate B-AC_{S}

The term “conjugate” primarily refers to covalent conjugates, such as chemical conjugates and recombinantly produced conjugates (where both the moieties and also a possible linker have peptide structure). The term also includes so-called native conjugates, i.e. affinity reactants exhibiting two binding sites that are spaced apart from each other, with affinity directed towards two different molecular entities, for instance a native antibody that comprises species and class-specific determinants on one side (=part) of the molecule and antigen/hapten-binding sites on another side (=part).

The conjugate may be present in the collection either predispensed to the microchannel structures of a device (i.e. to the interior of a device), or kept as a separate entity of the collection outside the devices. If predispensed, the conjugates may be present in the reaction microcavacities and affinity bound to the solid phase via the immobilized affinity ligand L, or in some other location within the microchannel structures/devices. If present outside the devices the conjugate is typically in a separate package as a solution or in dry form, e.g. lyophilized or dried at atmospheric or reduced pressure.

In an alternative embodiment the binder B in unconjugated form is part of the collection. The binder B is then typically delivered in a package that is separate from the devices, although one can envisage preloading it to the microchannel structures (i.e. to the interior of the devices) for preparing the conjugates within the devices at the time of use. Additional reagents that are necessary for preparing the conjugates, for instance activation reagents, are typically also separate from the devices in the collection.

In still another alternative the conjugate is provided from other sources, for instance synthesized by the customer.

The term in “unconjugated form” for binder B means a form that does not exhibit any binding site for the solute of interest. The term includes also binder B together with the reagents necessary for a conjugation with AC_{S}, and that B is in a form suitable for conjugation with AC_{S}. This typically means that B in unconjugated form in the context of the invention exhibits a functional group that is electrophilic or nucleophilic and is capable of forming covalent links with functional groups of an affinity reactant such as AC_{S}. Electrophilic, nucleophilic and functional groups can be selected, for instance, amongst amino groups and other groups comprising substituted or unsubstituted —NH₂, carboxy groups (—COOH/—COO⁻), hydroxy groups, thiol groups, keto groups etc. The structures discussed above for potentially being present in affinity reactants can also be utilized for conjugation if being present in either one or both of B and AC_{S}

Reaction Microcavity (104a-h) and Solid Phase

The reaction microcavity (104a-h) is defined as the part of a microchannel structure (101a-h) in which the solid phase is present. This means that for solid phases in the form of porous beds, the bed volume and the reaction microcavity (104a-h) will coincide and have the same volume. If the solid phase is the inner wall of a microconduit, the reaction microcavity (104a-h) is defined as the volume between the most upstream and the most downstream end of the solid phase.

The reaction microcavity (104a-h) has at least one cross-sectional dimension that is ≦1,000 μm, such as ≦500 μm or ≦200 μm (depth and/or width). The smallest cross-sectional dimension is typically ≧5 μm such as ≧25 μm or ≧50 μm. The volume of the reaction microcavity is typically in the nl-range, such as ≦5,000 nl, such as 1,000 nl or ≦500 nl≦100 nl or ≦50 nl or ≦25 nl.

The geometric form of reaction microcavities that contain a solid phase with affinity ligand L is typically essentially the same within a set but may or may not differ between the sets.

The solid phase is ideally the same within a set but may vary between the sets. One can envisage that in order to run experiments for certain selected solutes (e.g. analytes), concentration ranges, samples, protocols etc, optimal conditions will require selected solid phases that do not fit to other solutes, other concentration ranges, samples, protocols etc. Differences between solid phases may relate to the base matrix, such as porosity and/or Kav-values, composition of solid phase, presence or absence of pores permitting convective and/or diffusive mass transport of reactants, etc. Differences may also relate to immobilization techniques and the like.

The solid phase in a reaction microcavity may be a porous bed or one or more inner walls of the reaction microcavity. Examples of porous beds are a) a population of porous or non-porous particles that are packed to a bed, or b) a porous monolith.

The term “porous particles” have the same meaning as in WO 02075312 (Gyros AB).

Suitable particles may be spherical or spheroidal (beaded) or non-spherical. Suitable mean diameters for particles used as solid phases are typically found in the interval of 1-100 μm with preference for mean diameters that are ≧5 μm, such as ≧10 μm or ≧15 μm and/or ≦50 μm. Also smaller particles can be used, for instance with mean diameters down to 0.1 μm. The design of the outlet end (111a-h) of the reaction microcavity (104a-h) and the particles should match each other so that the particles can be retained in the reaction microcavity (104a-h). See for instance WO 02075312 (Gyros AB). Certain kinds of particles, in particular particles of colloidal dimension, may agglomerate. In these cases the size of the agglomerate should be in the intervals given even if the agglomerating particles as such are below. Diameters refer to the “hydrodynamic” diameters.

Particles to be used may be monodisperse (monosized) or polydisperse (polysized) in the same meaning as in WO 02075312 (Gyros AB).

The solid phase material may or may not be transparent.

The base material of a solid phase may be made of inorganic and/or organic material. Typical inorganic materials comprise glass, and typical organic materials comprise organic polymers. Polymeric materials comprise inorganic polymers, such as glass and silicone rubber, and organic polymers that may be of synthetic or biological origin (biopolymers). The term biopolymer includes semi-synthetic polymers in which there is a polymer backbone derived from a native biopolymer. Typical synthetic organic polymers are cross-linked and are often obtained by the polymerisation of monomers comprising polymerisable carbon-carbon double bonds. Examples of suitable monomers are hydroxy alkyl acrylates and corresponding methacrylates, acryl amides and methacrylamides, vinyl and styryl ethers, alkene substituted polyhydroxy polymers, styrene, etc. Typical biopolymers may or may not be cross-linked. In most cases they exhibit a carbohydrate structure, e.g. agarose, dextran, starch etc.

The term “hydrophilic” for porous beds means contemplates sufficient wettability of the surfaces of the pores for water to be spread by capillarity all throughout the bed when in contact with excess water (absorption). If the solid phase is the inner wall of a reaction microcavity “hydrophilic” primarily means that the water contact angle is within the limits specified for hydrophilicity (wettability) elsewhere in this specification. Alternatively the hydrophilicity is sufficient to fill up the reaction microcavity (104a-h) with water by capillarity once water has reached the most upstream end of the reaction microcavity. Surfaces that are to be in contact with aqueous liquids shall also expose a plurality of polar functional groups which each has a heteroatom selected amongst oxygen and nitrogen, for instance. Appropriate functional groups can be selected amongst hydroxy groups, eythylene oxide groups (—X—[CH₂CH₂O—]ₙ where n is an integer >1 and X is nitrogen or oxygen), amino groups, amide groups, ester groups, carboxy groups, sulphone groups etc, with preference for those groups that are essentially uncharged independent of pH, for instance within the interval of 2-12.

If the base material of a solid phase material is hydrophobic or not sufficiently hydrophilic, e.g. is based on a styrene (co)polymer, the surfaces that are to be in contact with an aqueous liquid may be hydrophilized. Typical protocols comprise coating with a compound or mixture of compounds exhibiting polar functional groups of the same type as discussed above, treatment by an oxygen plasma etc.

General Features of the Microfluidic Device

The number of microchannel structures containing the solid phase with affinity ligand L on the same microfluidic device is typically ≧10, e.g. ≧25 or ≧90 or ≧180 or ≧270 or ≧360. As discussed above the microchannel structures of a device may be divided in groups. The number of microchannel structures in each group is typically in the interval 1-99%, such as 5-50% or 5-25% or 10-50%, of the total number of microchannel structures of the device. This typically means that each group comprises 3-15 or 3-25 or 3-50 microchannel structures.

A microchannel structure (101a-h) of a microfluidic device comprises functional parts that permit the full protocol of an experiment to be performed within the structure. A microchannel structure (101a-h) may thus comprise one, two, three or more functional parts selected among: a) inlet arrangement (102,103a-h) comprising for instance an inlet port/inlet opening (105a-b,107a-h), possibly together with a volume-metering unit (106a-h,108a-h), b) microconduits for liquid transport, c) reaction microcavity (104a-h); d) mixing microcavity; e) unit for separating particulate matters from liquids (may be present in the inlet arrangement), f) unit for separating dissolved or suspended components in the sample from each other, for instance by capillary electrophoresis, chromatography and the like; g) detection microcavity; h) waste conduit/microcavity (112,115a-h), i) valve (109a-h,110a-h), j) vent to ambient atmosphere (116a-i), etc. A functional part may have more than functionality, e.g. reaction microcavity (104a-h) and a detection microcavity may coincide. Various kinds of functional units in microfluidic devices have been described by Gyros AB/Amersham Pharmacia Biotech AB: WO 99055827, WO 99058245, WO 02074438, WO 02075312, WO 03018198 (US 20030044322), WO 03034598, SE 03026507 (SE 04000717, U.S. Ser. No. 60/508,508), SE 03015393 (U.S. Ser. No. 60/472,924) and by Tecan/Gamera Biosciences: WO 01087487, WO 01087486, WO 00079285, WO 00078455, WO 00069560, WO 98007019, WO 98053311.

As already discussed for common inlet arrangements, the innovative microfluidic device may also comprise other common microchannels/micro conduits/microfluidic functions connecting two or more microchannel structures. Common channels including their various parts such as inlet ports, outlet ports, vents, etc., are considered part of each of the microchannel structures they are communicating with. In a similar manner as for the common inlet arrangements, these other common functions may be the basis for dividing the microchannel structures of a device into groups and/or designating different groups of the microchannel structures of a device to different subareas of a microfluidic device. These kind of groups or subareas do not need to coincide with the groups and subareas defined by common inlet arrangements.

Common microchannels make it possible to construe microfluidic devices in which the microchannel structures form networks. See for instance U.S. Pat. No. 6,479,299 (Caliper).

Each microchannel structure has at least one inlet opening for liquids and at least one outlet opening for excess of air (vents) and possibly also for liquids.

The microfluidic device of the innovative collection may also comprise microchannel structures that have no reaction microcavity for retaining a solid phase material according to the invention.

Different principles may be utilized for transporting the liquid within the microfluidic device/microchannel structures between two or more of the functional parts described above. Inertia force may be used, for instance by spinning the disc as discussed in the subsequent paragraph. Other useful forces are capillary forces, electrokinetic forces, non-electrokinetic forces such as capillary forces, hydrostatic pressure etc.

The microfluidic device typically is in the form of a disc. The preferred formats have an axis of symmetry (Cₙ) that is perpendicular to or coincides with the disc plane, where n is an integer ≧2, 3, 4 or 5, preferably ∞ (C_{∞}). In other words the disc may be rectangular, such as square shaped, and other polygonal forms but is preferably circular. Once the proper disc format has been selected centrifugal force may be used for driving liquid flow. Spinning the device around a spin axis that typically is perpendicular or parallel to the disc plane may create the necessary centrifugal force. In the most obvious variants at the priority date, the spin axis coincides with the above-mentioned axis of symmetry. Potentially important arrangements with spin axes that are non-perpendicular against a disc plane have been described (PCT/SE2003/001850 (Gyros AB).

For preferred centrifugal-based variants, each microchannel structure comprises one upstream section that is at a shorter radial distance than a downstream section (from the spin axis). A reaction microcavity that contains affinity ligand L is at a radial position intermediary to these two kinds of sections and is typically oriented with the flow direction radially outwards from the spin axis.

The preferred devices are typically disc-shaped with sizes and/or forms similar to the conventional CD-format, e.g. sizes that are in the interval from 10% up to 300% of a circular disc with the conventional CD-radii (12 cm). The upper and/or lower sides of the disc may or may not be planar.

Microchannels/microcavities of a microfluidic devices may be manufactured from an essentially planar substrate surface that exhibits the channels/cavities in uncovered form that in a subsequent step are covered by another essentially planar substrate (lid). See WO 91016966 (Pharmacia Biotech AB) and WO 01054810 (Gyros AB). Both substrates are preferably fabricated from plastic material, e.g. plastic polymeric material.

The fouling activity and hydrophilicity of inner surfaces should be balanced in relation to the application. See for instance WO 01047637 (Gyros AB).

The terms “wettable” (hydrophilic) and “non-wettable” (hydrophobic) contemplate that a surface has a water contact angle ≦90° or ≧90°, respectively. In order to facilitate efficient transport of a liquid between different functional parts, inner surfaces of the individual parts should primarily be wettable, preferably with a contact angle ≦60° such as ≦50° or ≦40° or ≦30° or ≦20°. These wettability values apply for at least one, two, three or four of the inner walls of a microconduit. In case one or more of the inner walls have a higher water contact angle this can be compensated for by a lower water contact angle for the remaining inner wall(s). The wettability, in particular in inlet arrangements should be adapted such that an aqueous liquid will be able to fill up an intended microcavity by capillarity (self suction) once the liquid has started to enter the cavity. A hydrophilic inner surface in a microchannel structure may comprise one or more local hydrophobic surface breaks in a hydrophilic inner wall, for instance for introducing a passive valve, an anti-wicking means, a vent solely function as a vent to ambient atmosphere etc (rectangles in FIG. 1). See for instance WO 99058245 (Gyros AB) and WO 02074438 (Gyros AB).

Contact angles refer to values at the temperature of use, typically +25° C., are static and measured by the method given in WO 00056808 (Gyros AB) and WO 01047637 (Gyros AB).

Packages of the Various Components of the Collections.

The microfluidic devices are typically delivered to the customer with the solid phase preloaded to the different reaction microcavities. The solid phase material in each reaction microcavity may be in a wet state but is preferably in a dry state (which includes also a dehydrated state) for future reconstitution by the customer. Solid phase material in dry state typically includes one or more agents that stabilize the solid phase material as such and/or stabilize the affinity ligand L from damages during a possible freezing, drying, storage and reconstitution (bed-preserving agents that includes cryostabilisators, lyostabilisators, stabilisators etc). See SE 03008232, U.S. Ser. No. 60/466376 and the corresponding International Patent Application filed in parallel with this application (“Pre-loaded microscale devices”) (Gyros AB) and Arakawa et al (Advanced Drug Delivery Reviews 46 (2001) 307-326). An important variant of bed-preserving agents is microcavity adherence agents. This kind of agents comprises a compound or a mixture of compounds that promote retaining of a solid phase material in a microcavity by increasing the adherence between the material and inner surfaces of the microcavity and/or between particles if the material is in particle form. A microcavity adherence agent typically comprises carbohydrate or polymer structure.

Other reagent components of the collection may also be provided in solution or in a dry state of the same kinds as discussed in the preceding paragraph. This applies for instance to the conjugate, the binder B in unconjugated form, other affinity reactants (if present) etc which when preloaded to the individual microchannel structures of the invention may be in a dry or a wet state. These components may be delivered in separate packages as part of a microfluidic device of the innovative collection or provided by the customer or some other supplier as discussed elsewhere in this specification.

Information about and/or manuals for the various sets of the innovative collection is typically included in sales material and material delivered to the customer, i.e. material that present function and/or use of one, two or more of the sets. This kind of material is also part of the innovative collection.

Second Aspect: The use of the Collection.

This aspect comprises a method for carrying out a plurality of runs of a particular protocol, in particular with the reaction between a particular solute S and its affinity counterpart AC_{S} in parallel. The steps of the method are:
(i) selecting the appropriate set in the innovative collection, and
(ii) selecting a microfluidic device comprising microchannel structures of the selected set,
(iii) providing a conjugate B-AC_{S} and a liquid containing the solute S,
(iv) sensitizing the solid phase in the reaction microcavities of at least a group of the microchannel structures of the set on the selected microfluidic device with the conjugate provided in step (iii),
(v) performing the affinity reaction betweeen the solute S by transporting an aliquot of the liquid through the microcavities of the subgroup, preferably by also including the other steps of the protocol, i.e. performing the complete protocol.

The various embodiments discussed in the context of the first aspect of the invention are applicable also to the second aspect. AC_{S} and S have been selected according to what has been outlined for these entities elsewhere in this specification.

Static Versus Flow Conditions in the Porous Bed During use.

The interaction between the immobilized affinity counterpart AC_{S} and the solute S may take place under static or flow conditions, i.e. with or without transport of the solute by a liquid flow passing through the reaction microcavities. We have previously found that more information may be gained under flow conditions than under static conditions (WO 02075312 (Gyros AB)). The flow rate and/or residence time may for instance be adjusted such that the amount of solute bound to the solid phase will reflect the actual reaction rate or affinity between an immobilized affinity reactant and a solute with a minimum of perturbation by diffusion (non-diffusion limiting conditions). This also applies to the present invention but does not exclude that for applications where the primary interest is the total amount of bound/captured solute, capturing under flow conditions utilizing either diffusion limiting or non-diffusion limiting conditions, or static conditions can be used. The appropriate flow rate through the porous bed depends on a number of factors, e.g. the immobilized reactant and the solute and their sizes, the volume of the reaction microcavity, the porous bed including the solid phase material etc. Typically the flow rate should give a residence time of ≧0.010 seconds such as ≧0.050 sec or ≧0.1 sec with an upper limit that typically is below 2 hours such as below 1 hour. Illustrative flow rates are within 0.01-1000 nl/sec, such as 0.01-100 nl/sec and more typically 0.1-10 nl/sec. These flow rate intervals may be useful for bed volumes in the range of 1-200 nl, such as 1-50 nl or 1-25 nl. Residence time refers to the time it takes for a liquid aliquot to be in contact with the solid phase in the reaction microcavity.

The Affinity Reaction

The affinity reaction between the solute S and its affinity counterpart AC_{S} may be part of an assay protocol or a separation protocol of the kind specified in the introductory part. In the most typical case the affinity reaction is part of an assay for the characterization of one or more reaction variables.

Reaction variables that are to be characterized by the use of affinity reactions are mainly of two kinds 1) variables related to affinity reactants, and 2) reaction conditions. The first category has two main subgroups a) amounts including presence and/or absence, concentration, relative amounts, activity such as binding activity and enzyme activity, etc, and b) properties of affinity reactants including affinity as such, e.g. affinity constants, specificities etc. The molecular entity for which a reaction variable of type 1 is characterized is called an analyte. See WO 02075312 (Gyros AB). The second category of reaction variables includes pH, temperature, ionic strength, presence of hydrogen bond breaking agents, detergents, liquid flow, immobilization techniques, solid phases etc for the affinity reaction studied. See WO 02075312 (Gyros AB).

The term “analyte” in the context of the present invention refers to an uncharacterized molecular entity that is present in a liquid sample and is to be characterized with respect to amount and/or to chemical or biochemical properties. The term includes analyte-derived entities that emanate from an original analyte in an original sample that has been processed to a sample used in a microfluidic device. This preprocessing may take place outside the microfluidic device and/or in separate substructures within the microfluidic device. Preprocessing may include that an original analyte participates in affinity reactions, enzymatic and/or chemical conversion etc. The analyte-derived entity may have a chemical composition that is different from the original analyte, be an affinity complex or an uncomplexed affinity reactant that differ from the original analyte etc. The presence and amount of the analyte-derived entity is always related to the occurrence of the original analyte in the original sample. The solute S may be an analyte-derived entity or the original analyte of the original sample.

The assay reaction studied concerns formation or dissociation of an affinity complex. The characterization typically involves: a) Determination of an uncharacterized amount of an analyte in a sample, b) Selection of binders (analytes) from a library of potential binder candidates, c) Determination of immobilization techniques and/or solid phases that are optimal for a given affinity pair, D) Determination of suitable reaction conditions related to the liquid in which the reaction is carried out, e) Determination of a ligand and/or a binder with respect to their suitability for the dissociation of their affinity complex, and f) Determination of qualitative aspects of complex formation, etc. See WO 02075312 (Gyros AB) for further details.

Major Assay Protocols.

In a typical protocol an uncharacterized amount of an analyte of a sample is allowed to form an affinity complex comprising at least the analyte and an affinity counterpart to the analyte (anti-analyte). Depending on the protocol used, additional affinity reactants may be used and possibly also incorporated into the complex. The amount and type of reactants are selected so that the affinity reactions involved will result in an amount of the affinity complex that will reflect the true binding ability and/or the amount of the analyte in the sample. See for instance WO 02075312 (Gyros AB).

These assay protocols typically utilize an analytically detectable reactant and/or an immobilized or immobilizable reactant. Either one or both of these reactants are fully or partly incorporated into the complex comprising the analyte. Immobilization is used to separate the complex comprising the analyte from reactants not incorporated in the complex, for instance to separate a complex comprising an analytically detectable reactant from uncomplexed forms of the same reactant.

There are mainly two kinds of assay protocols that can be applied to the innovative collection of microfluidic devices.

Competitive/Inhibition Protocols.

In these protocols the analyte and an analyte analogue are competing with each other for binding to a limiting amount of an anti-analyte. The anti-analyte may be
(a) immobilized or immobilizable if the analyte analogue is soluble and analytically detectable, and
(b) analytically detectable if the analyte analogue is immobilized or immobilizable.

For variant (a) and depending on the exact protocol used, the immobilized or immobilizable anti-analyte may correspond to affinity counterpart AC_{S} and the analyte and/or the analyte analogue to solute S. For variant (b), the immobilized or immobilizable analyte analogoue may correspond to affinity counterpart AC_{S} and the analytically detectable anti-analyte analogue to solute S.

Among competitive variants, alternative (b) has been of great interest for the invention during the initial development phase up to now. This variant includes that the analyte and the anti-analyte are preincubated before reaching the reaction microcavity (104a-h), for instance outside the microfluidic device or in a separate mixing microcavity upstream the reaction microcavity. The mixture is transported through the reaction microcavity (104a-h) where the free (=uncomplexed) anti-analyte forms an affinity complex with an immobilized analyte analogue. This complex is subsequently measured. The uncomplexed anti-analyte may be regarded as solute S and the immobilized analyte analogue then corresponds to affinity counterpart AC_{S}.

Competitive variants include displacement assays in which an immobilized or immobilizable affinity complex comprising an analyte analogue and an anti-analyte is incubated with an analyte (=solute).

Non-Competitive Protocols

These protocols typically utilize non-limiting amounts of one or more affinity counterparts to the analyte.

The most important non-competitive protocols are of the sandwich-type and comprise formation of immobilized or immobilizable complexes in which an analyte is sandwiched between two anti-analytes (that are equal or analogues to each other). One of the anti-analytes is analytically detectable and the other immobilized or immobilizable and possibly also analytically detectable. Depending on the exact protocol used the immobilized or immobilizable anti-analyte may correspond to affinity counterpart AC_{S}, The analyte, or the complex between the analyte and the other anti-analyte analyte, or the uncomplexed form of the other anti-analyte may then correspond to solute S.

Another non-competitive variant utilizes only one anti-analyte, which is in immobilized or immobilizable form. In this case complex formation leads to an immobilized complex, or a soluble complex that subsequently is immobilized. The immobilized complex as such is then measured. In a typical variant the anti-analyte corresponds to affinity counterpart AC_{S} and the analyte to solute S.

Analytically Detectable Reactants.

By the term “analytically detectable” is contemplated an affinity reactant that can be analytically discriminated from other affinity reactants participating in the formation of the complex to be measured. Detectability may derive from an inherent property of the reactant, for instance an inherent biological function such as the enzyme activity of an enzyme or Fc-receptor binding activity of various Ig-classes and subclasses, or a separately introduced functionality, e.g. labeling with an analytically detectable tag or label, such as biotin (=affinity label), enzyme, chromogen, fluorogen, fluorophor, chemiluminscent group, radioactive group etc.

The formed complex as such may also be detectable, for instance by changing the optic properties of a solution, of a surface etc.

A detectable label may be combined with a second label selected such that the labels together give the appropriate signal when the complex is formed or dissociated. This variant may be illustrated with so-called scintillation proximity assays (SPA) and by using pairs of interacting fluorophores (FRET).

### Experimental Part

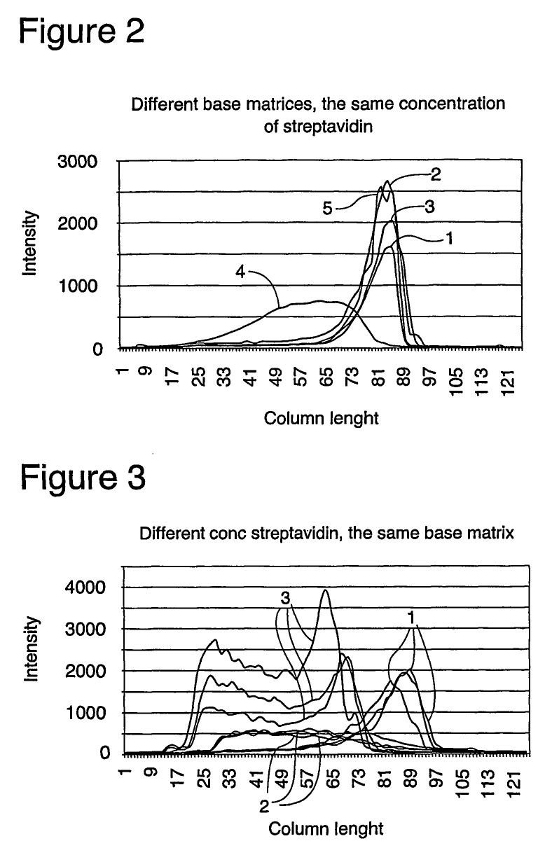

The microfluidic device used for the experiments was circular and of the same dimension as a conventional CD (compact disc). This microfluidic device will further on be called CD. The CD contained 14 groups (100) of 8 microchannel structures (101a-h) arranged in an annular zone around the center (spin axis) of the disc with a common waste channel (112) for each group (100) close to the periphery. A group of 8 microchannel structures is shown in FIG. 1 and is similar to and function in the same manner as the subgroup illustrated in FIGS. 1-2 of (WO 02075312, Gyros AB) and the corresponding figures in WO 03024548 (US 20030054563) (Gyros AB) and WO 03024598 (US 20030053934) (Gyros AB). The dimensions are essentially of the same size as in these earlier patent applications.

Each subset (100) comprises eight microchannel structures (101a-h) with one common inlet arrangement (102), one separate inlet arrangements (103a-h) per microchannel structure, and onet reaction microcavity (104a-h) per microchannel structure. The common inlet arrangement comprises a) two common inlet ports (105a-b) that also will function as outlet ports for excess liquid, and b) one volume-metering unit (106a-h) for each microchannel structure (101a-h). The volume-metering units (106a-h) will function as a distribution manifold for the downstream parts of the microchannel structures. Each of the separate inlet arrangements (103a-h) is part of only one microchannel structure and comprises an inlet port (107a-h) and a volume-metering unit (108a-h). Between each volume-metering unit (106a-h, 108a-h) and their downstream parts, respectively, there is a valve function (109a-h, 110a-h), preferably passive. A reaction microcavity (104a-h) of a microchannel structure (101a-h) is located downstream both the common inlet arrangement (102) and a separate inlet arrangement (103a-h) of a microchannel structure (101a-h). At the outlet end (111a-h) of each reaction microcavity, the depth is lowered from 100 μm to 10 μm in two steps to prevent particles from escaping the reaction microcavity. Each reaction microcavity (104a-h) is in the downstream direction connected to an outlet microconduit (113a-h) that in FIG. 1 is illustrated as an outward bent and has an outlet end (114a-h) connected to a waste function (115a-h). At the periphery there is a common waste channel (112). Vents (116a-i, hydrophobic breaks) together with the valves (109a-h, hydrophobic breaks) define the volume of the liquid aliquots to be distributed downstream from each the volume-metering unit (106a-h).

By applying the appropriate volume of aqueous liquid to the inlet port of an inlet arrangement, capillarity will fill the volume-metering unit(s) connected to the inlet port with liquid. By spinning the disc around its center, liquid can be forced to pass the valve (109a-h,110a-h) between a volume-metering unit and downstream parts.

Experimentals

Instrumentation

The immunoassay was performed in an automated system. The system (Gyrolab Workstation, prototype 2 instrument equipped with a Laser Induced Fluorescence (LIF) module, Gyros AB, Uppsala, Sweden) was equipped with a CD-spinner, holder for microtiter plates (MTP) and a robotic arm with a holder for 10 capillaries connected to 5 syringe pumps, 2 and 2. Two of the capillaries transferred all the reagents and buffers from a MTP to either of the two common inlet ports (105a-b) in the CD. The other eight capillaries transferred individual samples from a MTP to the separate individual inlet ports (107a-h) in the CD.

Gyrolab Workstation is a fully automated robotic system controlled by application-specific software. An application specific method within the software controls the spinning of the CD at the precisely controlled speeds and thereby controls the movement of liquids through the microstructures as the application proceeds. Special software was included in order to reduce background noise.

See also WO 02075312 (Gyros AB), WO 03025548 and US 20030054563 (Gyros AB), WO 03025585 and US 200030055576 (Gyros), WO 03056517 and US 200301156763 (Gyros AB) and also www.gyros.com.

Preparation of Porous Beds.

For example solid polystyrene (PS) particles (15 μm, Dynal Biotech, Oslo, Norway) were selected for the solid phase. The beads were modified by passive adsorption of phenyl-dextran (PheDex) to create a hydrophilic surface and were subsequently covalently coupled with streptavidin (Immunopure Streptavidin, Pierce, Perbio Science UK Limited, Cheshire, United Kingdom) using CDAP chemistry (Kohn & Wilchek, Biochem. Biophys. Res. Commun. 107 (1982), 878-884). Solid phase material in the form of porous particles (beads) were Superose™, Superdex™ Peptide (Superdex™ P) and Sepharose™ HP (Amersham Biosciences, Uppsala, Sweden) have also been covalently coupled with streptavidin using CDAP chemistry (without phenyl-dextran coating). The polystyrene particles are solid and non-swellable, and Superose™, Superdex™ Peptide and Sepharose™ HP are porous in relation to many affinity reactants and swellable in the liquids used.

After coupling with streptavidin, a suspension of the particles in potassium phosphate buffer (10 mM) was distributed in the common distribution channel (102) via inlet port (105a-b) and moved through the structure by centrifugal force. The centrifugal force combined with the vents (109a-h,113a-i) divide the suspension in the common inlet arrangement (102) in equal portions, each of which forms a bed of packed particles (column) in each reaction microcavity (104a-h) against the dual depth (111a-h). The approximate volume of the column was 15 nl.

Immunoassay

The catching antibody in our myoglobin assay, the monoclonal anti-myoglobin 8E11.1 (LabAS, Tartu, Estonia) was biotinylated using Sulfo-NHS-LC-biotin (Pierce, prod # 21335, Perbio Science UK Limited, Cheshire, United Kingdom). The protein concentration of the monoclonal antimyoglobin 8E11.1 was 1-10 mg/ml. The anti-myoglobin 8E11.1 was incubated in room temperature for 1 h with 3× molar excess of the biotinylation reagent in 15 mM PBS with 0.15 M NaCl before the reaction mixture was gel filtrated through either a NAP-5 column (Amersham Biosciences, Uppsala, Sweden) or a Protein Desalting Spin Column (Pierce, # 89849-P, Perbio Science UK Limited, Cheshire, United Kingdom).

To load the streptavidin immobilized particles with the biotinylated antibody, a solution at a 0.2-2 mg/ml concentration (depending of how much streptavidin it is in the packed column) of antibody was distributed in the common distribution channel via inlet port (105a-b) and moved through the structure by centrifugal force. The flow rate through the columns was controlled by the spin velocity (spin flow 1). After the capturing antibody was attached to the columns they were washed once by addition of PBS (with 0.01% Tween™ 20) to the common distribution channel (inlet ports 105a or b) followed by a spin step.

The myoglobin samples (diluted in PBS with 1% BSA) of 500 nM each were distributed to the individual inlet ports (107a-h). The sample volume 200 nl was defined into the volume-metering unit (108a-h), during the first two steps in the spin flow method. To reach favourable kinetic conditions under the capturing step (for myoglobin to bind to 8E11.1) the flow rate of the sample should not exceed 1 nl/sec. The sample flow rate was controlled by spin flow 2. After sample capturing the columns was washed twice by addition PBS (with 0.01% Tween 20) to the common distribution channel (inlet port 105a or b) followed by a spin step. Detecting antibodies (monoclonal antimyoglobin 2F9.1 (LabAs, Tartu, Estonia)) in excess were applied next via the common distribution channel (inlet port 105a or b) and a similar slow flow rate (spin flow 3) was used. The detecting antibodies were labeled with a fluorophore Alexa 633 (Molecular Probes, Eugene, USA). Excess of labeled antibody was washed away by 4 additions of PBS (with 0.01% Tween 20) to the common distribution channel (inlet port 105a or b). Each addition was followed by a spin step. Washing with aqueous isopropanol (IPA) significantly reduced non-specific adsorption.

The complete assay was analyzed in the Laser Induced Fluorescence (LIF) detector module. See more WO 02075312 (Gyros AB), WO 03025548 and US 20030054563 (Gyros AB), ), WO 03025585 and US 200030055576 (Gyros AB), and WO 03056517 and US 200301156763 (Gyros AB).

An overview of the run method performed in the system is presented in Table 1.

Results

FIG. 2b illustrates that solid phases comprising different base matrices could be used. The only base matrix that deviates for the concentrations used is PS-PheDex. FIG. 3 illustrates that solid phase material comprising the same base matrix but with different concentrations of immobilized affinity ligand L (streptavidin in this case). The solid phase comprising the larger binding capacity is tentatively used when lower concentrations of analyte are to be measured (higher sensitivity). The unwantedly bound material appearing downstream the main peak for (graph 3) could be removed if a wash with aqueous isopropanol (IPA) was included in the protocol.

TABLE 1

| METHOD | SPIN PROFILE |
|---|---|
| Rewetting of bead columns | |
| Spin 1 | 2500 rpm 5 s, 6000 rpm 10 s |
| Wash of beads | |
| Spin 2 | 1200 rpm 2 s, 2500 rpm 0.5 s, 4000 rpm 10 s, |
| | 6500 rpm 16 s |
| Transfer of biotinylated antibody | |
| Spin flow 1 | 1200 rpm 2 s, 2500 rpm 0.5 s, from 1200-1500 rpm |
| | 45 s, 2000 rpm 35 s, 3000 rpm 30 s, |
| | 4000 rpm 10 s, 5000 rpm 5 s, 6000 rpm 10 s |
| Wash of beads and CD-structure 1 | |
| Spin 3 | 1200 rpm 2 s, 2500 rpm 1 s, 4000 rpm 15 s, |
| | 6000 rpm 18 s |
| Transfer of myoglobin samples | |
| Spin flow 2 | 1000 rpm 5 s, 2500 rpm 0.5 s, from 1200-1500 rpm |
| | 90 s, 2000 rpm 70 s, 3000 rpm 60 s, |
| | 4000 rpm 20 s, 5000 rpm 10 s |
| Myoglobin wash 1 | |
| Spin 4 | 1200 rpm 2 s, 2500 rpm 0.5 s, 4000 rpm 10 s, |
| | 6000 rpm 16 s |
| Myoglobin wash 2 | |
| Spin 5 | 1200 rpm 2 s, 2500 rpm 0.5 s, 4000 rpm 10 s, |
| | 6000 rpm 16 s |
| Transfer of conjugate | |
| Spin flow 3 | 1200 rpm 2 s, 2500 rpm 0.5 s, from 1200-1500 rpm |
| | 90 s, 2000 rpm 70 s, 3000 rpm 60 s, |
| | 4000 rpm 20 s, 5000 rpm 10 s |
| Conjugate wash 1 | |
| Spin 6 | 1200 rpm 2 s, 2500 rpm 0.5 s, 4000 rpm 10 s, |
| | 6000 rpm 16 s |
| Conjugate wash 2 | |
| Spin 7 | 1200 rpm 2 s, 2500 rpm 0.5 s, 4000 rpm 10 s, |
| | 6000 rpm 16 s |
| Conjugate wash 3 | |
| Spin 8 | 1200 rpm 2 s, 2500 rpm 0.5 s, 4000 rpm 10 s, |
| | 6000 rpm 16 s |
| Conjugate wash 4 | |
| Spin 9 | 1200 rpm 2 s, 2500 rpm 0.5 s, 4000 rpm 10 s, |
| | 6000 rpm 16 s |
| Detection | |

Drying and Reconstitution

Certain innovative aspects of the invention are defined in more detail in the appending claims. Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

1. A collection of one or more microfluidic devices which together carry a plurality of microchannel structures each of which comprises a reaction microcavity in which there is a solid phase with an immobilized affinity ligand L,
wherein:
(i) the plurality comprises two or more different sets of microchannel structures, and
(ii) the affinity ligand L is directed to the same counterpart (binder, B) independent of set, and
(iii) the sets differ with respect to
a) the capacity for binder B per reaction microcavity and/or the capacity per unit volume of the solid phase in a reaction microcavity, and/or
b) the base matrix of the solid phase
between the sets but are equal within each set.

2. The collection according to claim 1, wherein the difference is with a factor ≧1.2 for at least one of the sets of the collection compared to the binding capacity for the set having the lowest binding capacity.

3. The collection according to claim 1, wherein at least one of said devices comprises
a) at least two of said sets of microchannel structures, and/or
b) only one set of microchannel structures, with the proviso that the collection comprises two or more devices which are different with respect to the kind of sets they carry.

4. The collection according to claim 1 being intended for separately performing one or more affinity protocols that differ with respect to the reactants involved and/or the order of addition of the reactants and/or the concentration range in which the reactants are used, each of said different protocols utilizing an affinity reaction between
(i) a solute S, and
(ii) a conjugate comprising
(a) a binder B, and
(b) an affinity counterpart AC_{S} to the solute S,
wherein the affinity constant K_{L-B} for formation of the complex L-B between the affinity ligand L and the binder B, i.e. K_{L-B}=[L][B]/[L-B], is at most 10³ times, such as at most 10² times, the corresponding affinity constant for streptavidin and biotin.

5. The collection according to claim 4, wherein L is selected amongst biotin-binding compounds and streptavidin-binding compounds, respectively, or vice versa.

6. The collection of claim 4, wherein L has two or more binding sites for B.

7. The collection according to claims 1, wherein
(a) that each set on a device is grouped into one or more groups of fluidly equivalent microchannel structures, and
(b) that each group is located to a particular subarea of the device.

8. The collection according to claims 1, wherein said reaction microcavity in at least one, preferably all, of said microchannel structures in the upstream direction is connected to a volume-metering unit.

9. The collection according to claim 7, wherein said volume-metering unit is part of an inlet arrangement for liquid.

10. The collection according to claims 6, wherein said volume-metering unit within at least one of said group(s) are part of a distribution manifold for distributing liquid to the reaction microcavities of the group, with the proviso that each of said at least one group comprises two or more microchannel structures.

11. The collection according to claims 7, wherein the inner wall of each of said volume-metering units have a sufficient hydrophilicity for said unit to filled by capillarity once an aqueous liquid have entered the unit, and b) a valve at its outlet end, for instance a passive valve.

12. The collection according to claims 4, wherein at least one of the solute S and its affinity counterpart AC_{S}, and/or at least one of the binder B and the ligand L comprise a structure selected from the group of peptide structure consisting of poly/oligo-peptide and protein structure, carbohydrate structure, lipid structure including steroid structure, nucleotide structure including nucleic acid structure, and polymeric structure.

13. The collection according to claims 1, wherein said solid phase is in a dry state, preferably comprising in addition to the solid phase one or more bed-preserving agents.

14. The collection according to claim 13, wherein at least one of said one or more bed-preserving agents is a microcavity adherence agent.
